(19) United States
(12) Patent Application Publication
(10) Pub. No.: US 2025/0057490 A1
(43) Pub. Date: Feb. 20, 2025

(54) METHOD FOR RECORDING PROJECTION IMAGES FROM DUAL ENERGY IMAGING AND X-RAY FACILITY

(71) Applicant: Siemens Healthineers AG, Forchheim (DE)

(72) Inventor: Alois Regensburger, Poxdorf (DE)

(21) Appl. No.: 18/807,965

(22) Filed: Aug. 17, 2024

(30) Foreign Application Priority Data
Aug. 17, 2023 (DE) 10 2023 207 915.1

(51) Int. Cl. A61B 6/03; A61B 6/00; A61B 6/40

(52) U.S. Cl. CPC A61B 6/032; A61B 6/4021; A61B 6/4441

(57) ABSTRACT

A method for recording projection images from dual energy imaging is provided. At least one pair of mutually associated projection images of an examination object is recorded with different X-ray spectra. For the recording of each pair, two focal spots of an X-ray radiator spaced from one another in a movement plane are used. A first projection image is recorded in a first radiator position of the X-ray radiator using a first X-ray spectrum originating from a first focal spot. The X-ray radiator is moved into a second radiator position such that a second focal spot comes to lie at least within a tolerance range about the position of the first focal spot in the first radiator position. A second projection image is recorded in the second radiator position of the X-ray radiator using a second X-ray spectrum originating from the second focal spot.

This application claims the benefit of German Patent Application No. DE 10 2023 207 915.1, filed on Aug. 17, 2023, which is hereby incorporated by reference in its entirety.

### BACKGROUND

The present embodiments relate to recording projection images from dual energy imaging.

In X-ray dual energy imaging, projection images are recorded using different X-ray spectra, but usually using the same projection geometry. The X-ray spectra may include, for example, a high energy spectrum and a low energy spectrum. Possible image processing measures for such pairs of projection images includes the, for example, linear and/or weighted combination of the projection images. Dual energy imaging may also be used for computed tomography (CT) in which from low-dimensional projection images of different projection geometries (e.g., different projection angles in a circular recording trajectory of the X-ray radiator), higher-dimensional image datasets are reconstructed. Herein also, initially, image processing of the pairs of projection images takes place in many application cases before the reconstruction is performed.

Since, during computed tomography, the X-ray radiator (and often also the X-ray detector) are moved continuously along the recording trajectory, particular challenges arise for the dual energy imaging variants that successively record the projection images of different X-ray spectra.

In principle, different variants of the dual energy imaging exist. First, the different X-ray spectra may be generated one after the other with an X-ray radiator, and the projection images may be recorded. Successive different spectral filters may also be used (e.g., the spectral filters be introduced into the beam path). It has therein been proposed to use techniques for rapidly switching over the tube voltage and/or for rapidly changing filters in order to generate temporally successive X-ray pulses of the different X-ray spectra during the movement at least of the X-ray radiator. Herein, however, there exists an offset between the two projection images related, for example, to the rotation, which makes the evaluation more difficult and generates additional artifacts.

In the prior art, a number of approaches to solving this problem have previously been proposed. For example, two-layer detectors are capable of recording projection images for two different X-ray spectra in one shot, so that the problem of the rotation offset does not arise. However, the problem of poorer spectral separation does then occur. In addition, a lower dose efficiency follows.

With photon-counting detectors also, it is possible to record projection images for two X-ray spectra in one shot; however, for example, with regard to the use of an X-ray facility with a C-arm system, such detectors are expensive and present challenges for their production.

It has also been proposed to undertake two separate successive recording procedures along the same recording trajectory, where the recording trajectory is traveled once using the first X-ray spectrum and once using the second X-ray spectrum. However, this approach is extremely sensitive with regard to movements of the examination object (e.g., a patient and to deformations of the anatomy). When a C-arm is used, problems may arise with regard to the exact repeatability of the recording trajectory. Finally, very long breath holding is needed. This approach is therefore only usable in at least almost static situations (e.g., in neurology).

If, however, a rapid change of the X-ray spectra (e.g., a rapid switch-over of the tube voltage and/or a rapid change of the filter) is used in order to record temporally sequential pairs of projection images during the rotation, it has also first been proposed to undertake a three-dimensional image reconstruction, optionally with a model-based and/or iterative approach, in order to avoid a direct combination or joint image processing of the projection images of the pairs (e.g., pixel-wise). Also conceivable are separate reconstructions for the X-ray spectra in order thereafter to bring the results together. In addition or alternatively, a 2D movement compensation between the two projection images may take place. However, an appreciable movement of the focus of the X-ray radiator takes place between the recording of the projection images, for example, by a number of millimeters (e.g., several millimeters). Therefore, the movement compensation cannot be optimal since the X-rays have taken a physically different path through the examination object.

In another context, a concept has been proposed in which two different focal spots are used in spiral CT, in the direction perpendicular to the rotation direction (e.g., longitudinal direction of the spiral, z-direction) with the same X-ray spectra in order ultimately to sample two offset spirals. In this way, the resolution in the longitudinal direction of the spiral may be increased, the dose efficiency may be increased, and spiral artifacts, for example, in neurological computed tomography, may be reduced. Purely by way of example, reference is made to the presentation “Double Z Sampling: How Does It Work and What Does It Do?” by M. R. Bruesewitz et al., RSNA 2005, retrievable under https://www.mayo.edu/research/documents/rsna2005-doublez-samplingpdf/DOC-10027306.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an advantageously applicable variant for the recording of projection images by dual energy imaging, for example, in the context of computed tomography is provided.

A method provides that, for the recording of each pair of mutually associated projection images, two focal spots of the X-ray radiator spaced from one another in the movement plane (e.g., the rotation plane) are used. A first projection image of the pair is recorded in a first radiator position of the X-ray radiator using a first X-ray spectrum originating from a first focal spot. By moving (e.g., rotating) the recording arrangement in the movement plane (e.g., the rotation plane), the X-ray radiator is moved into a second radiator position such that the second of the focal spots comes to lie at least within a tolerance range about the position of the first focal spot in the first radiator position. A second projection image of the pair is recorded in the second radiator position of the X-ray radiator using a second X-ray spectrum originating from the second focal spot.

Herein, the X-ray radiator moves, for example, along a circular path in the projection plane. For example, on recording of just one pair, a correction of the position of the X-ray radiator in the projection direction may also be provided. However, in principle, other recording trajectories along which the X-ray radiator may move (e.g., continuously) may also be provided to record a plurality of pairs of projection images.

In one embodiment, in a recording procedure (e.g., a computed tomography recording), a plurality of pairs of mutually associated projection images are recorded for different projection geometries along a single recording trajectory of the X-ray radiator (e.g., at a constant movement velocity of the X-ray radiator).

The present embodiments may be applied for computed tomography investigations of an examination object (e.g., a patient). The X-ray radiator (e.g., also the X-ray detector) is moved continuously along a recording trajectory. The recording trajectory is therein at least substantially circular and/or covers a projection angle region of 180° plus the fan-beam arc. Therein, an X-ray facility with a C-arm on which the X-ray radiator and the X-ray detector are arranged opposite one another may be used as the X-ray facility. Alternatively, the X-ray facility may also be a computed tomography facility in which the X-ray radiator and the X-ray detector are guided in a gantry.

However, other application fields with other forms of the recording trajectory may, in principle, also be used (e.g., tomosynthesis, such as of the female breast, in which linear recording trajectories may be provided, and the second focal spot may even adopt exactly the position of the first focal spot). However, the present embodiments relate mainly to circular recording trajectories, but without being restricted thereto.

A fundamental idea of the present embodiments is therefore the use of two focal spots of the X-ray radiator that, however, are offset in the movement direction (e.g., with the rotation movement perpendicular to the projection direction (the direction of the central ray) and to the rotation axis). The focal spots are physically displaced from one another on the X-ray radiator, so that, therefore, the focus spacing between the focal spots also exists during the movement of the X-ray radiator in the also moving coordinate system (e.g., of the X-ray radiator). Each of the focal spots is used for emitting one of the two X-ray spectra. Herein, the concrete recording parameters (e.g., the physical spacing of the focal spots (focus spacing) and/or the movement velocity, such as the rotation velocity (angular velocity of the X-ray radiator)) and/or the temporal pause between the X-ray pulses of the energy spectra are selected such that due to the forward movement of the X-ray radiator, at the time point of the output of the X-ray pulse of the second energy spectrum from the second focal spot, the second focal spot is situated at least substantially at the location (e.g., in the fixed coordinate system relating to the examination object) at which the first focal spot was situated at the time point of the output of the X-ray pulse of the first energy spectrum. In the fixed (e.g., not also moving coordinate system (of the X-ray facility/the examination object)), the second focal spot therefore comes to lie, via the movement of the X-ray radiator for image recording, at the position of the first focal spot (e.g., for its image recording).

Herein, the known techniques for rapid switching over of the focal spot as are known, for example, from the described z-oversampling in spiral CT, and of the X-ray spectrum (e.g., the rapid switching over of the tube voltage and/or the rapid changing of the filter), as is known, for example, from the CT techniques using one focal spot, may be utilized.

Therefore, rapid focus switching is used to generate two temporally sequential and spatially separated focus positions, for example, on a rotating anode of the X-ray radiator. At each focal spot, a different X-ray spectrum is generated (e.g., by using different tube voltages and/or spectral filters). The two focal spots are positioned such that the two focal spots lie along the movement direction, for example, of the rotation direction. The time sequence of the emission of the X-ray pulses in context with the other recording parameters (e.g., also the focus spacing) is selected such that the deviation between the projection geometries of the two focal spots at the recording time points is small (e.g., minimal or at least almost eliminated).

The X-ray radiator (e.g., including an X-ray tube), therefore, has the capability of changing between two focal spots in temporal sequence and also of selecting the X-ray spectra used in a focal spot-specific manner, for example, by adapting the tube voltage. For example, in the case of an X-ray radiator having a rotating anode, it may be provided that two defined paths on the rotating anode are used for the focal spots. In order to generate the electron beams for the different focal spots, different emitters and/or field generators may be used for generating the electric fields influencing the movement path of the electrons. Alternatively or additionally, it may be provided, for example, in the use of two paths on the rotating anode, to provide the two paths on the rotating anode with different materials.

One of the key points of this present embodiments is the targeted selection of the recording parameters (e.g., the relationship between the position of the focal spots, the movement direction, and the X-ray pulse time sequence).

Such a concept with which changing focal spots are used during a recording procedure (e.g., during the continuous movement of the X-ray radiator along a recording trajectory) is also known as a “flying focal spot.” In this respect, the concept of the present embodiments may be described as a flying focal point with different X-ray spectra in the movement direction. The movement offset (e.g., the rotation offset, the angular offset) between temporally adjacent X-ray pulses of the two different X-ray spectra for generating a pair of X-ray images is reduced or eliminated. This provides that, despite the further continuous movement (e.g., rotation) of the X-ray radiator in the coordinate system of the examination object (e.g., a patient or a phantom), a pair of successive X-ray pulses of different X-ray spectra (e.g., a high energy spectrum and a low energy spectrum) is generated at at least approximately the same physical location in the fixed coordinate system (e.g., of the X-ray facility/the examination object).

In this way, a simpler dual energy evaluation is achieved for the dual energy imaging (e.g., for computed tomography; with a C-arm). Computed tomography with a C-arm in conical beam geometry is therein also designated cone beam computed tomography (CBCT).

As another example, the best possible matching projection geometries for the projection images of a pair is obtained. For example, during a rotation movement and a typical embodiment of X-ray radiators and X-ray detectors, an exact matching of the positions of the focal spots at the respective recording time points is not possible. If the best possible matching is aspired to, it may, however, be provided, as is considered in greater detail below, to pursue subsidiary goals.

In general, it has proved to be suitable if the tolerance range, naturally in the fixed coordinate system, is defined such that the spacing of the second focal spot in the second radiator position from the first focal spot in the first radiator position is less than, for example, 35% or less than 10% of the movement path of the X-ray radiator between the first radiator position and the second radiator position. In this way, for example, in comparison with variants having fast switching of the X-ray spectrum when using a single focal spot and continuous movement, a marked improvement is already achieved as far as the joint processing capability of the projection images of the pair is concerned. The matching of the positions of the focal spots has proved therein to be more important in the movement direction, for example, during the rotation, than in the projection direction, so that it may be provided that the spacing of the second focal spot in the second radiator position from the first focal spot in the first radiator position in the movement direction (e.g., the rotation direction) is smaller than in the projection direction (e.g., not more than half as large). For example, it may be provided that the spacing of the second focal spot in the second radiator position from the first focal spot in the first radiator position in the movement direction is less than 5%.

In a development of the present embodiments, it may be provided that during continuous movement with an at least partially constant movement velocity (e.g., an at least partially constant rotation velocity) of the X-ray radiator and a predetermined focus spacing of the focal spots of the X-ray radiator, a temporal spacing of the output of the X-ray pulses for the first projection image and the second projection image of each pair is selected such that the second focal spot comes to lie at least within the tolerance range about the position of the first focal spot in the first radiator position. For example, the desired location of the positions of the focal spots close to one another at the respective recording time points may thus be achieved in that with a specified movement velocity and a specified focus spacing, the time sequence (e.g., the timing) of the X-ray pulses is suitably selected. Therein, further parameters may also be utilized. For example, in the context of the rotation, herein, the spacing of the X-ray radiator from the isocenter of the X-ray facility (e.g., the mid-point about which the rotation takes place) is also used.

This will now be described in greater detail using an example. If a spacing of the focal spots of the X-ray radiator of 650 mm from the isocenter is assumed, the length of the circular recording trajectory over, for example, 200° is 2270 mm. During such a rotation, for example, a total of 500 projection images (e.g., 250 pairs) are to be recorded. If the focus spacing is 4.5 mm, with a known rotation velocity, a sequence of recording time points may be established, so that the X-ray radiator moves by this 4.5 mm between two projection images, at least within the tolerance range.

In general, it may be stated for the rotation movement that the focus spacing, the spacing of the X-ray radiator (or specifically the focal spots) from the isocenter, the rotation velocity, and the temporal spacing between the pulses are matched to one another, such that the second of the focal spots comes to lie at least within the tolerance range about the position of the first focal spot in the first radiator position.

In one embodiment, it may be provided that the recording parameters defining the movement (e.g., the rotation movement) of the recording arrangement and/or the recording parameters defining the recording of the first projection image and the second projection image and/or the focus spacing in an optimization procedure are selected such that the spacing of the second focal spot in the second radiator position from the first focal spot in the first radiator position is minimized. In this embodiment, therefore, an optimum solution is defined, where in more complex circumstances, optimization algorithms may also be utilized. Herein, boundary conditions may also be formulated, depending upon which variables form exactly the parameters to be optimized. In general, a suitable embodiment may provide that in the optimization procedure, it is required as a boundary condition that the displacement of the X-ray detector in the image plane of one of the projection images via the movement of the recording arrangement corresponds to a whole number multiple of a pixel size of pixels of the X-ray detector. In this way, the movement (e.g., also completed) of the X-ray detector between the recording of the projection images that leads to a displacement of image information items that correspond to one another, in the coordinate system of the X-ray detector, may be taken into account particularly easily.

Before this is considered in greater detail, it should be noted that, in general, suitably, whenever the region of the examination object covered by the radiation field changes due to the movement of the X-ray detector, a comparison of image contents within each pair takes place such that the same portions of the examination object are shown. Specifically, it may be provided that due to the movement of the recording apparatus, image contents are discarded in only one of the projection images of a pair (e.g., at least for a common evaluation of the projection images of the pair).

For the preparation of a common evaluation of each pair of projection images, in order to compensate for the movement of the X-ray detector, it may further be provided that the projection images of each pair are displaced against one another by the displacement of the X-ray detector in the image plane of one of the projection images. For example, during a joint rotation movement of the X-ray radiator and the X-ray detector, as the X-ray radiator is moved at least substantially by the focus spacing, the X-ray detector also moves further. The displacement herein occurring may be compensated for via a corresponding displacement of one of the projection images such that the same pixel corresponds at least substantially to the same ray path. A particularly simple correction possibility arises if, as described above, the displacement of the X-ray detector in the image plane via the movement of the recording apparatus corresponds to a whole number multiple of a pixel size of pixels of the X-ray detector. In other cases, more complex (e.g., interpolating) algorithms may be used.

During the rotation movement, a tilting of the X-ray detector may occur. However, during computed tomography and other applications where the projection images of a pair are recorded shortly after one another via rapid switching over, the tilting is so small that the tilting needs no special correction (e.g., if the tilting between the recording of the projection images is less than 0.5°).

Nevertheless, embodiments may provide, for example, with tilting angles greater than 0.5°, that on a rotation movement, by applying a compensation algorithm to each pair of projection images, a tilting of the X-ray detector due to the rotation movement is also compensated for. In other words, for example, more complex image distortion methods may be applied in order to compensate also for the rotation (e.g., usually small rotation) of the X-ray detector.

In example embodiments, a general compensation of the position change of the detector may be undertaken by applying a registration algorithm.

As previously mentioned, a general evaluation of a pair of projection images may provide, for example, that from the projection images of each pair, in an evaluating procedure, via, for example, linear and/or weighted and/or pixel-wise combination of the projection images, an evaluation image is established. For example, a pixel-wise calculation takes place, which may be simplified by corresponding prior compensation for the displacement of the X-ray detector. The pixel-wise combination may involve, for example, a subtraction, although other (e.g., weighted) linear combinations may be provided.

In a development of the present embodiments, it may be provided that for at least one of the focal spots of the X-ray radiator, a spectral filter of the X-ray radiator is applied. Using a spectral filter, the X-ray spectrum may be brought to the desired form in an improved manner. Although procedures for the rapid changing of filters are known in principle, such procedures are particularly suitable to provide the spectral filter fixedly on the X-ray radiator such that the spectral filter acts only upon the corresponding focal spot. Specifically, therefore, it may be provided that the at least one spectral filter is fixedly associated with the respective focal spot (e.g., that the at least one spectral filter is unalterably installed in the X-ray radiator). For example, a fixedly installed spectral filter may be provided on an exit window of the X-ray radiator or may extend in annular manner on the rotating anode, rotating with the rotating anode.

In a development, for example, also with regard to the X-ray facility, it may be provided that an X-ray radiator that is rotatable about the projection direction is used. For recording the projection images, the X-ray radiator is rotated or remains such that the focal spots follow one another in the movement direction (e.g., the rotation direction). This enables, for example, with the same X-ray facility, other applications also to be performed in which the focal spots are to follow one another in other directions. It may thus be provided that, in a second rotation position, the focal spots follow one another perpendicularly to the projection direction and the rotation direction in a z-direction (e.g., longitudinal direction), so that projection images displaced relative to one another in the z-direction may be recorded, for example, using the same X-ray spectrum and/or in the context of a spiral CT. Corresponding investigation possibilities have already been described in the introduction. Thus, an X-ray facility that is usable in varied ways is obtained.

As already mentioned, apart from the method, the present embodiments also relate to an X-ray facility having a recording arrangement that includes an X-ray radiator and an X-ray detector opposite one another (e.g., on a C-arm and movable jointly in a movement plane, such as rotatable in a rotation plane), and a control facility (e.g., a controller) that is configured for carrying out a method according to the present embodiments. All the embodiments relating to the method according to the present embodiments may be transferred similarly to the X-ray facility according to the present embodiments with which the above-mentioned advantages may therefore also be achieved.

For example, the control facility has at least one memory storage device and at least one processor. Via hardware and/or software, functional units may be formed in order to implement acts of the method according to the present embodiments. Specifically, the control facility may include, for example, a basically known recording unit that may control the recording activity of the recording arrangement and its movement. For example, if, for the recording of each pair of mutually associated projection images, two focal spots of the X-ray radiator mutually spaced in the movement plane are used, the recording unit may therefore also carry out the recording of the first projection image in the first radiator position using the first focal spot and the recording of the second projection image in the second radiator position using the second focal spot with corresponding first and second X-ray spectra and also the movement of the recording arrangement in the movement plane of the X-ray radiator into the second radiator position, such that the second of the focal spots comes to lie about the position of the first focal spot in the first radiator position at least within a tolerance range. The control facility may further have an establishing unit for the recording parameters (e.g., the X-ray pulse time sequence), such that, when the recording parameters are used, the corresponding recording sequence for each pair of projection images comes about. Further, an image processing unit may be provided (e.g., for the compensation of the detector movement between the recording time points of the projection images of a pair). Herein, for example, the displacement (e.g., “shifting”) is corrected, as described above. An evaluating unit for joint evaluation (e.g., combination) of the pairs of projection images may also be provided. With regard to a rotatability of the X-ray radiator (e.g., also a z-offset of projection images), a setting unit may also be provided for rotating the X-ray radiator.

The method according to the present embodiments may also be implemented by a computer program that has program means that, when the computer program is carried out in a control facility of an X-ray facility, cause the control facility to carry out the acts of a method according to the present embodiments. The computer program may be stored on an electronically readable data carrier (e.g., a non-transitory computer-readable storage medium) that therefore includes control information stored thereon. The control information includes at least one computer program of this type and is configured such that, on use of the data carrier in a control facility of an X-ray facility, the facility is configured to carry out a method according to the present embodiments. The data carrier may be, for example, a non-transient data carrier (e.g., a CD-ROM).

### BRIEF DESCRIPTION OF THE DRAWINGS

FIG. 1 shows a flow diagram of an example embodiment of a method;

FIG. 2 shows a projection geometry for recording a first projection image of a pair;

FIG. 3 shows a projection geometry for recording a second projection image of a pair;

FIG. 4 one embodiment of an X-ray facility; and

FIG. 5 shows the functional structure of a control facility of the X-ray facility.

### DETAILED DESCRIPTION

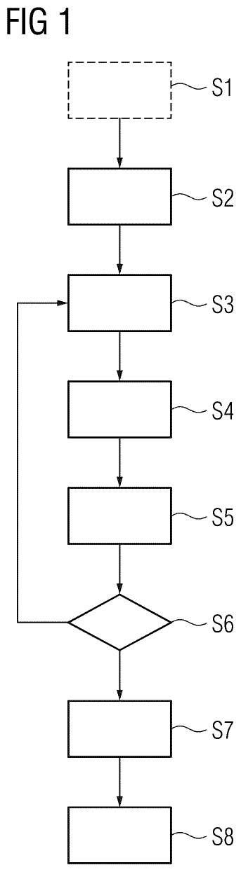

FIG. 1 shows a flow diagram of an example embodiment of a method for recording pairs of projection images of a dual energy imaging with an X-ray facility (e.g., an X-ray device). Therein, in the present case, by way of example, a computed tomography scan is carried out (e.g., with an X-ray facility having a C-arm (cone beam CT (CBCT)). An X-ray radiator of the X-ray facility moves continuously at a particular movement velocity (e.g., an angular velocity) along a circular recording trajectory that may cover, for example, 200° or, in general, 180° plus a fan-beam arc. During this rotation movement of the X-ray radiator, a plurality of (e.g., 250) pairs of projection images are recorded, of which a first projection image is to be recorded with a first X-ray spectrum (e.g., a low energy spectrum) and a second projection image is to be recorded with a second X-ray spectrum (e.g., a high energy spectrum). Therein, the example embodiment described here offers the possibility, despite continuous movement of the X-ray radiator and a time offset between the X-ray pulses for the two X-ray spectra, of a similar (e.g., at least substantially identical) projection geometry for the first projection image and the second projection image.

For this purpose, in the present case, an X-ray radiator is used, with which two separate focal spots that are arranged offset in a movement plane in which the recording arrangement is movable (e.g., rotatable; in the movement direction, such as the rotation direction) may be used. The focus spacing in the coordinate system (e.g., also moving coordinate system) of the X-ray radiator and the recording parameters (e.g., therefore the parameters describing the movement) and the X-ray pulse time sequence are matched to one another, such that the second focal spot at the time of the output of the X-ray pulse for the second projection image (e.g., second radiator position), relative to the fixed coordinate system of the examination object (or the X-ray facility), is situated at least within a tolerance range about the position of the first focal spot at the time point of the output of the X-ray pulse for the first projection image of the pair (e.g., first radiator position). For this purpose, suitable recording parameters may already be specified, but optionally, also, for example, if other recording parameters may be selected in a targeted manner, established in act S1. Therein, it is, for example, the temporal spacing between the output of the X-ray pulse for the first projection image and the X-ray pulse for the second projection image (e.g., the recording time point) that is adapted dependent upon the other specified parameters. Therefore, the X-ray pulse time sequence is suitably selected or stipulated during a continuous rotation movement along the recording trajectory.

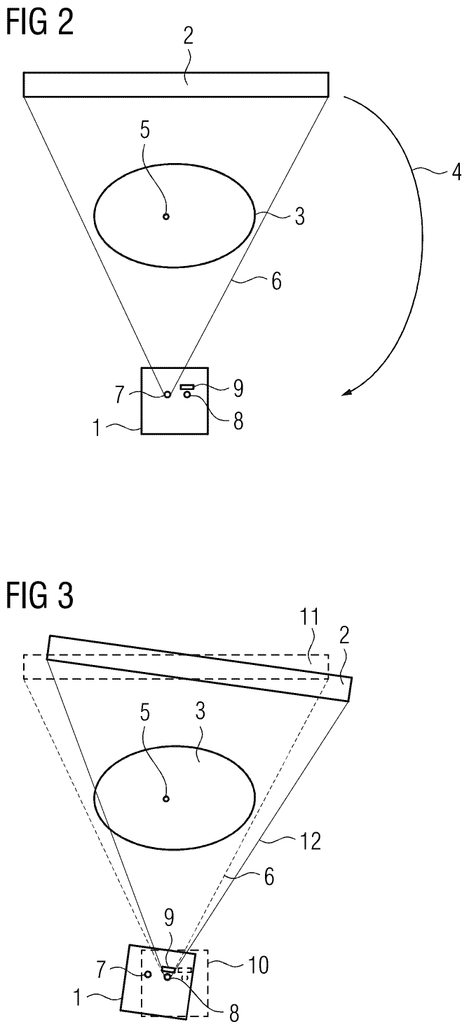

FIGS. 2 and 3 illustrate the fundamental concept of the present embodiments in the form, in each case, of a sketch of the principle of the first radiator position and the second radiator position.

FIG. 2 shows schematically the recording arrangement of the X-ray facility with the X-ray radiator 1 and the X-ray detector 2 arranged opposite thereto. Arranged between the X-ray radiator 1 and the X-ray detector 2 is the examination object 3 (e.g., a patient or a phantom). The recording arrangement is rotatable, according to the arrow 4, about an isocenter 5 of the X-ray facility in the image plane of FIGS. 2 and 3 as the movement plane. Also indicated is the beam field 6 that is output in a cone beam geometry in this first radiator position that emerges from a first focal spot 7 of the X-ray radiator 1. Therein, a first X-ray spectrum is used.

As FIG. 2 shows, however, the X-ray radiator 1 also has a further, second focal spot 8 physically spaced from the first focal spot 7 in the coordinate system of the X-ray radiator 1 that is also moved during the movement of the X-ray radiator 1. The second focal spot is offset relative to the first focal spot 7 in the movement plane (e.g., the movement direction of the rotation). The two focal spots 7, 8 that are spaced from one another by the focus spacing (e.g., by 3 to 6 mm) may be realized, for example, by different focal point paths on a rotating anode of the X-ray radiator 1. In the present case, a fixed spectral filter 9 is also associated with the second focal spot 8. The filter is also able to be provided on the rotating anode (e.g., annular) but is also able to cover a corresponding portion of the exit window of the X-ray radiator 1 used by the second focal spot 8. In order to generate the different X-ray spectra, apart from the use of spectral filters 9, the use of different emitters and/or different materials on the rotating anode may also be utilized. The filter may also be configured to be rapidly exchangeable.

FIG. 3 shows the second radiator position in which the recording arrangement of the X-ray radiator 1 and the X-ray detector 2 has rotated further according to the arrow 4. Therein, it should first be noted that herein, for representation reasons, a significantly too large rotation angle of 6° is used since the rotation angles typically specified based on the possibility for rapid switching over between the focal spots 7, 8 and X-ray spectra are significantly smaller (e.g., less than 0.5°) and are not suitable for representation in a clear manner.

For better intelligibility, FIG. 3 also shows, dashed, the positions 10 and 11 of the X-ray radiator 1 and of the X-ray detector 2 from FIG. 2, as well as the beam field 6. In the second radiator position of FIG. 3, starting from the second focal spot 8 that is visibly situated close to or even on the position of the first focal spot 7 in the first radiator position according to FIG. 2 in the fixed coordinate system, the radiation field 12 is formed. Additionally, the filter 9 is used. Further, the image recording takes place here with the second X-ray spectrum.

In other words, the focus spacing and the recording parameters (e.g., the X-ray pulse time sequence) are selected so that the recording of the first projection image takes place with the first X-ray spectrum from the first focal spot 7 in the first radiator position and the recording of the second projection image takes place with the second X-ray spectrum from the second focal spot 8 in the second radiator position with at least approximately the same projection geometry. By way of the rotation, the second focal spot 8 is inserted into the previous position of the first focal spot 7, relative to the fixed coordinate system of the examination object 3 and the X-ray facility.

In the second radiator position in the fixed coordinate system, the second focal spot 8 lies at least within a tolerance range in the fixed coordinate system about the position of the first focal spot 7 in the first radiator position. The tolerance range may therein be defined in that the spacing of the second focal spot 8 in the second radiator position from the first focal spot 7 in the first radiator position (e.g., in the fixed coordinate system) is less than 35% (e.g., less than 10%) of the movement path of the X-ray radiator 1 between the first radiator position and the second radiator position (e.g., in the fixed coordinate system), which is taken here as the reference. Since therein the comparability of the projection directions is more important, the spacing of the second focal spot in the second radiator position from the first focal spot in the first radiator position in the rotation direction in the fixed coordinate system is less than in the projection direction. In other example embodiments, it may be provided that the second focal spot assumes exactly the position of the first focal spot (e.g., in tomosynthesis).

During the establishing or specifying of the recording parameters, for example, the X-ray pulse time sequence that may take place in an optimization procedure, apart from a minimization of the spacing of the position of the second focal spot in the second radiator position and of the first focal spot in the first radiator position, boundary conditions may also be taken into account (e.g., to the effect that a displacement of the X-ray detector 2), as is readily apparent in FIG. 3 by a multiple of the width of a pixel (e.g., pixel detector) of the X-ray detector 2 takes place. This is described in greater detail below.

Returning to FIG. 1, in act S2, the general recording procedure begins. This provides that the X-ray radiator 1 and the X-ray detector 2 begin their continuous rotation movement making use of the recording trajectory, and the X-ray pulse time sequence is used in order to define the recording time points of the projection images of the plurality of pairs. Herein, the X-ray spectra alternate constantly, where the pauses between the individual pairs of projection images do not necessarily have to correspond to the temporal spacing between the projection images of a pair, but rather may be shorter or, for example, longer.

For a pair of projection images, where the first projection image is recorded with the first X-ray spectrum using the first focal spot 7 and the second projection image is recorded using the second focal spot 8 as close as possible to the previous position of the first focal spot 7, first, in act S3, the first projection image is recorded at the first radiator position. Thereafter, in act S4, for example, without further actuating measures being necessary, since it is continuous, the further movement of the X-ray radiator 1 (e.g., and parallel therewith, the X-ray detector 2) into the second radiator position takes place. After expiry of a corresponding temporal spacing that is suitably specified or established, in act S5, the second projection image is recorded with the second X-ray spectrum from the second focal spot 8, as close as possible to the position of the first focal spot 7 in the first radiator position.

In act S6, it is checked whether the recording procedure is now ended or if further pairs of projection images are to be recorded. If the latter is the case, a return to act S3 may take place where, after the expiry of a time spacing between pairs as specified by the X-ray pulse time sequence, the next first projection image is recorded. Otherwise, the recording procedure is ended.

In act S7, an image processing of the recorded pairs takes place such that at least the displacement of the X-ray detector 2 from the first radiator position into the second radiator position is corrected in order to be able to perform pixel-wise calculations on the projection images of a pair in an uncomplicated manner. For example, if the displacement of the X-ray detector 2 takes place by a multiple of the pixel size, this may be easily achieved. For example, if the X-ray detector 2 has been tilted by more than 0.5° from the first radiator position to the second radiator position, a compensation for this tilting may also be provided. In any case, image contents that are contained in only one of the projection images of a pair are withdrawn from the further evaluation and are discarded.

The projection image pairs prepared in this way are then evaluated in act S8, where in the context of the evaluation, the projection images of a pair are combined pixel-wise, weighted linearly, to at least one evaluation image, which based on the use of similar or even identical projection geometries and the image processing in act S7 is possible without difficulty and with useful results.

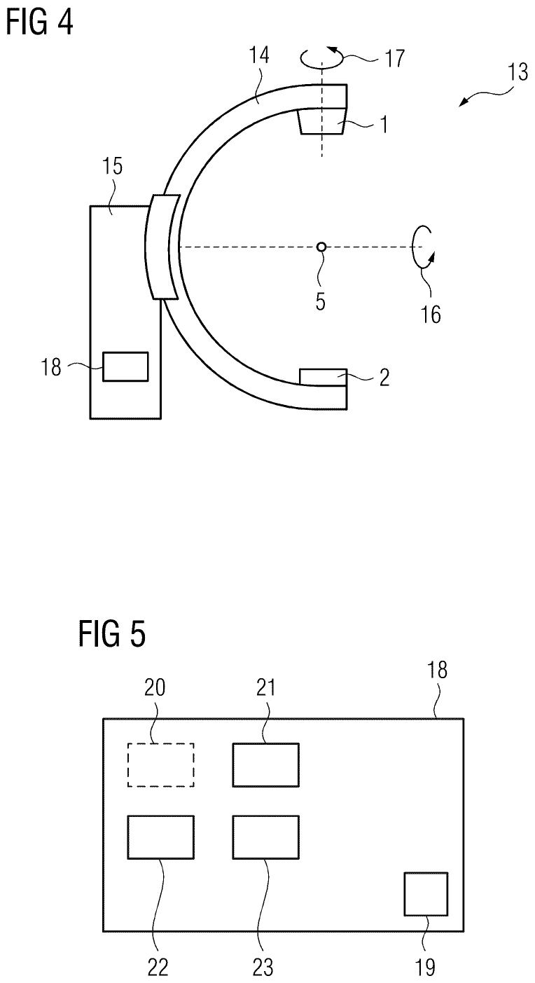

FIG. 4 shows a sketch of the principle of an example embodiment of an X-ray facility 13. The X-ray facility 13 has a C-arm 14 on which the X-ray radiator 1 and the X-ray detector 2 are arranged opposite one another. The C-arm 14 is held on a stand 15 and is rotatable according to the arrow 16 at least about a rotation axis extending through an isocenter 5.

The X-ray radiator 1 may also be arranged rotatably on the C-arm 14 according to the arrow 17. Herein, a corresponding rotary actuator that is drivable by the control facility 18 of the X-ray facility 13 may be provided. The rotatability of the X-ray radiator 1 permits the focal spots 7, 8 also to follow one another in directions other than the rotation direction, for example, along a longitudinal direction (e.g., z-direction) in the image plane of FIG. 4. Then, for example, spiral CT recordings with a flying focal spot may be recorded. For the method of FIG. 1, in act S2 before the beginning of the recording procedure, a backward rotation is to take place such that the focal spots 7, 8 follow one another in the rotation direction again.

Naturally, further degrees of freedom of movement may also be provided in the X-ray facility 13.

FIG. 5 shows the functional structure of the control device 18 in greater detail. The control device 18 has a storage device 19, for example, for storing recording parameters, projection images, and suchlike. An optional establishing unit 20 may be provided for carrying out the optional act S1.

In a recording unit 21, the recording operation of the X-ray facility 13 is controlled (e.g., also, the movement taking place during the recording procedure). The recording unit 21 therefore serves, for example, for carrying out the acts S2 to S6. In an image processing unit 22, the image processing according to act S7 may take place, where the evaluation of the act S8 may take place in an evaluating unit 23. With regard to the rotation of the X-ray radiator 1 about its own axis according to the arrow 17, an adjusting unit (not shown here in detail) may also be provided.

Although the invention has been illustrated and described in detail via the example embodiments, the invention is not restricted by the examples disclosed, and other variations may be derived therefrom by a person skilled in the art without departing from the protective scope of the invention.

Independent of the grammatical term usage, individuals with male, female, or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for recording projection images from dual energy imaging, wherein at least one pair of mutually associated projection images of an examination object is recorded with different X-ray spectra, and an X-ray facility with a recording arrangement is used, the X-ray facility comprising an X-ray radiator and an X-ray detector that are jointly movable opposite one another and in a movement plane, wherein, for the recording of each pair of mutually associated projection images, two focal spots of the X-ray radiator spaced from one another in the movement plane are used, the method comprising:
recording a first projection image of the at least one pair in a first radiator position of the X-ray radiator using a first X-ray spectrum originating from a first focal spot of the two focal spots;
moving the recording arrangement in the movement plane, such that the X-ray radiator is moved into a second radiator position, such that a second focal spot of the two focal spots comes to lie at least within a tolerance range about a position of the first focal spot in the first radiator position; and
recording a second projection image of the at least one pair in the second radiator position of the X-ray radiator using a second X-ray spectrum originating from the second focal spot.

2. The method of claim 1, wherein, in a recording procedure, a plurality of pairs of mutually associated projection images are recorded for different projection geometries along a single recording trajectory of the X-ray radiator.

3. The method of claim 2, wherein the recording procedure is a computed tomography recording, and
wherein, in the recording procedure, the plurality of pairs of mutually associated projection images are recorded for different projection geometries along the single recording trajectory of the X-ray radiator at an at least partially constant rotation velocity of the X-ray radiator.

4. The method of claim 1, wherein the tolerance range is defined such that a spacing of the second focal spot in the second radiator position from the first focal spot in the first radiator position is less than 35% of a movement path of the X-ray radiator between the first radiator position and the second radiator position, such that that the spacing of the second focal spot in the second radiator position from the first focal spot in the first radiator position in a movement direction is smaller than in the projection direction, or a combination thereof.

5. The method of claim 4, wherein the tolerance range is defined such that the spacing of the second focal spot in the second radiator position from the first focal spot in the first radiator position is less than 10% of the movement path of the X-ray radiator between the first radiator position and the second radiator position, such that the spacing of the second focal spot in the second radiator position from the first focal spot in the first radiator position in a rotation direction is not more than half as large than in the projection direction, or a combination thereof.

6. The method of claim 1, wherein during continuous movement with an at least partially constant movement velocity of the X-ray radiator and a pre-determined focus spacing of the two focal spots of the X-ray radiator, a temporal spacing of an output of X-ray pulses for the first projection image and the second projection image of each pair of the at least one pair is selected such that the second focal spot comes to lie at least within the tolerance range about the position of the first focal spot in the first radiator position.

7. The method of claim 1, wherein recording parameters defining the movement of the recording arrangement, recording parameters defining the recording of the first projection image and the second projection image, a focus spacing, or any combination thereof in an optimization procedure is selected such that a spacing of the second focal spot in the second radiator position from the first focal spot in the first radiator position is minimized.

8. The method of claim 7, wherein in the optimization procedure, it is required as a boundary condition that a displacement of the X-ray detector in an image plane of one projection image of the first projection image and the second projection image via the movement of the recording arrangement corresponds to a whole number multiple of a pixel size of pixels of the X-ray detector.

9. The method of claim 1, wherein, in order to compensate for the movement of the X-ray detector, the projection images of each pair are displaced against one another by displacement of the X-ray detector in an image plane of one of the projection images.

10. The method of claim 9, further comprising on a rotation movement, applying a compensation algorithm to each pair of the at least one pair of mutually associated projection images, such that a tilting of the X-ray detector due to the rotation movement is also compensated for.

11. The method of claim 1, further comprising establishing an evaluation image from the projection images of each pair of the at least one pair, in an evaluating procedure, via linear combination, weighted combination, pixel-wise combination, or any combination thereof of the projection images.

12. The method of claim 1, further comprising applying a spectral filter of the X-ray radiator for at least one of the two focal spots of the X-ray radiator.

13. The method of claim 1, wherein the X-ray radiator is an X-ray radiator that is rotatable about a projection direction that, for recording the projection images, is rotated or remains such that the two focal spots follow one another in a movement direction.

14. An X-ray facility comprising:
a recording arrangement comprising:
an X-ray radiator and an X-ray detector arranged opposite one another and movable jointly in a movement plane; and
a controller configured to record projection images from dual energy imaging,
wherein at least one pair of mutually associated projection images of an examination object is recordable with different X-ray spectra, wherein, for the recordation of each pair of mutually associated projection images, two focal spots of the X-ray radiator spaced from one another in the movement plane are used, the controller being configured to record the projection images from dual energy imaging comprising the controller being configured to:
record a first projection image of the at least one pair in a first radiator position of the X-ray radiator using a first X-ray spectrum originating from a first focal spot of the two focal spots;
move the recording arrangement in the movement plane, such that the X-ray radiator is moved into a second radiator position, such that a second focal spot of the two focal spots comes to lie at least within a tolerance range about a position of the first focal spot in the first radiator position; and
record a second projection image of the at least one pair in the second radiator position of the X-ray radiator using a second X-ray spectrum originating from the second focal spot.

15. The X-ray facility of claim 14, wherein the X-ray radiator and the X-ray detector are arranged on a C-arm.

16. The X-ray facility of claim 14, wherein the X-ray radiator and the X-ray detector are rotatable jointly in a rotation plane.
